(19) United States
(12) Patent Application Publication
(10) Pub. No.: US 2018/0214361 A1
(43) Pub. Date: Aug. 2, 2018

(54) HAIR COMPOSITION

(71) Applicant: Conopco, Inc., d/b/a UNILEVER, Englewood Cliffs, NJ (US)

(72) Inventors: Hideyuki KOJOMA, Yokohama City (JP); Masahiro KASAI, Utsunomiya-city (JP); Christopher John ROBERTS, Spital, Wirral (GB); Jiro SANO, Kawasaki-city (JP); Nozomi WADA, Kawasaki-city (JP)

(73) Assignee: Conopco, Inc., d/b/a UNILEVER, Englewood Cliffs, NJ (US)

(21) Appl. No.: 15/747,531

(22) PCT Filed: Jul. 28, 2016
(86) PCT No.: PCT/EP2016/068033

§ 371 Date: Jan. 25, 2018

(30) Foreign Application Priority Data
Aug. 4, 2015 (EP) 15179697.6

(51) Int. Cl. A61K 8/31; A61Q 5/12; A61K 8/37; A61K 8/81; A61K 8/891; A61K 8/898

(52) U.S. Cl. CPC A61K 8/31; A61Q 5/12; A61K 8/898; A61K 8/8111; A61K 8/891; A61K 8/37

(57) ABSTRACT

A hair treatment composition, comprising, by weight of the total composition:—a) from 60 to 80 wt % isododecane, b) from 0.5 to 10 wt % isononyl isononanoate, and c) from 5 to 30 wt % of hydrogenated polyisobutene.

### FIELD OF INVENTION

This invention relates to hair treatment compositions to their use in the treatment of hair.

### BACKGROUND AND PRIOR ART

Volatile cyclic silicones are used extensively in hair compositions as carriers or solvents for other silicones high molecular weight oils. This enables the provision of hair compositions that provide a balance between sensory and performance attributes. The volatile cyclic silicones facilitate the spreading of the high molecular weight species on the hair, which in turn alleviates sticky feeling during and after product use.

Consumers desire conditioning such that their hair feels soft and moisturised. Dry, crispy or sticky feel negatively impact the conditioned feel of hair.

We have found that some volatile cyclic silicone materials, such as cyclopentasiloxane, present problems with spreading efficiency and sticky feel.

In addition, regulation and environmental pressures have caused formulators to look for alternative technologies.

There is a need for alternative systems that are able to deliver a high level of consumer performance, without loss of conditioned feel.

JP 2014/001206 (Shiseido) discloses an oil for both hair and body comprising (a) 10 to 50 wt % of a low-viscosity ester oil with a viscosity of 100 mPa s or less, (b) 10 to 30 wt % of a volatile hydrocarbon oil, (c) 1 to 15 wt % of a vegetable oil, (d) 10 to 20 wt % of a non-volatile oil and (e) 1 to 10 wt % of a low-viscosity silicone.

EP 2392314 A (Kokyu Alcohol Kogyo Co) discloses an oily hair cosmetic comprising an ester compound of a dibasic acid and one or more oils. The product is said have a high improving effect on damaged hair and excellent feeling upon use.

We have now found that a carrier system based on a combination of specific hydrocarbon based materials (isododecane, isononyl isononanoate and hydrogenated polyisobutene), rather than silicones, provides excellent spreadability and reduced stickiness on hair and hands.

### DEFINITION OF THE INVENTION

Accordingly in a first aspect the present invention there is provided a hair treatment composition, comprising, by weight of the total composition:
a) from 60 to 80 wt % isododecane,
b) from 0.5 to 10 wt % isononyl isononanoate, and
c) from 5 to 30 wt % of hydrogenated polyisobutene.

The invention further relates to a method of treating hair comprising the step of contacting hair with the composition of the first aspect.

A further aspect of the invention is a use of a composition as defined in aspect 1 to provide reduced stickiness of hand during use.

### DETAILED DESCRIPTION OF THE INVENTION

Compositions of the invention comprise, by weight of the total composition from 60 to 80 wt %, preferably from 65 to 75 wt %, isododecane,

from 0.5 to 10 wt %, preferably from 0.75 to 8 wt %, more preferably from 1 to 5 wt %, most preferably from 1 to 3 wt % of isononyl isononanoate, and

from 5 to 30 wt %, preferably from 5 to 15 wt % of hydrogenated polyisobutene.

Preferably, the wt:wt ratio of isododecane:isononyl isononanoate is in the range of from 120:1 to 8:1.

The compositions of the invention preferably further comprise at least one silicone.

A preferred silicone has a MWt in the range of from 500,000 cs to 20,000,000 cs, preferably 1000,000 cs to 15,000,000. Preferably, said silicone is present in an amount of from 1 to 15 wt %, preferably from 1 to 10 wt % by weight of the total composition. A suitable example is KF-9030 supplied by Shin-Etsu Silicones, which consists of 20% active high molecular weight polymerised Dimethicone (having a mean molecular weight 3,000,000 cs) blended with 20 cs low viscosity Dimethicone.

Another preferred silicone has a MWt in the range of from 50,000cs-499,999cs, preferably from 75,000-350,000 cs). Preferably, said silicone is present in an amount of from 5 to 15 wt %, by weight of the total composition. A suitable material is DC 200 (300,000 cs) also supplied as Xiameter PMX-200 or SH 200, which is supplied by Dow Corning Silicones. This consists of 100% Dimethicone Fluid with an average kinematic viscosity of 300,000 cs.

The compositions of the invention may comprise an amino modified silicone. Where present, the amino modified silicone is present in an amount of from 0.01-5 wt %, preferably 0.01-1 wt %, by weight of the total composition. A suitable example is DC 2-8566 which is supplied by Dow Corning Silicones, which consists of 100% Amino-modified Dimethicone Fluid.

Preparation Process

The compositions of the invention may be made by the following preferred process of preparation:

Isododecane, Hydrogenated Polyisobutylene and optional silicone fluid (300,000 cs) e.g. SH 200, are added to a vessel and stirred until a homogenous mixture is formed.

Optionally, silicone gum blend e.g. KF-9030, and amino modified silicone fluid e.g. DC 2-8566 are added and stirred until a homogenous mixture is obtained.

Isononoyl Isononanoate is added and the mixture stirred until homoegenous.

Further ingredients such as fragrance are then added, if required, with stirring.

Any polar and aqueous based ingredients may be incorporated using a suitable solubilising material. This mixture should then be added to the main vessel and stirred until a homogenous mixture is formed.

Product Format

Preferred product forms are leave on formulations, preferably selected from serums and oils.

It is preferred if the products are left on hair after application and not immediately washed off (within 1 hour of application). Such products are known as leave in compositions (also referred to herein as leave on compositions).

Such products frequently include further additional components. The additional components required to formulate such products vary with product type and can be routinely chosen by one skilled in the art. The following is a description of some of these carriers and additional components.

Further Ingredients

The composition of the invention may comprise further adjuncts suitable for use in application to hair.

Such adjuncts include conditioning materials such as quaternary silicone polymers, silicone based conditioners and their emulsions, and amino functional silicones.

Silicone copolyols and silicone polyethers, such PEG-8 dimethicone, may be added, preferably in an amount of from 0.1 to 2 wt %, by weight of the total composition, to enable emulsification of additional aqueous materials, for example, natural extracts. Preferred natural extracts include amino acids, proteins, sugars and ceramides and are preferably present in an amount of from 0.001 to 1.0 wt %, by weight of the total composition.

Other preferred adjuncts include oils, preferably naturally derived oils such as plant oils and vegetable oils, preferably those derived from nuts and seeds. Some suitable examples are coconut oil, sunflower oil, palm oil, olive oil and almond oil.

Further preferred adjuncts are fragrances, dyes, sunscreens (preferably benzophenone and methoxycinnamate derivatives), vitamins (for example, vitamin E acetate) and antioxidants (for example, butylated hydroxyl toluene (BHT)).

The Method

The method of the invention comprises applying compositions of the invention to dry or wet hair. Where the hair is wet, application is preferably followed by natural or fan assisted drying.

The following non-limiting Examples further illustrate the preferred embodiments of the invention. All percentages referred to in the examples and throughout this specification are by weight based on total weight unless otherwise indicated.

### EXAMPLES

### Example 1

### Compositions 1, 2 and 3, in Accordance with the Invention, and Comparative Composition A

Compositions 1, 2 and 3 were made, in accordance with the invention. A comparative example, designated A, was made which comprised cyclopentasiloxane as carrier.

Compositions 1, 2, 3 were made according to the following protocol:
1. Isododecane, hydrogenated polyisobutylene and silicone fluid (SH 200) were added to a vessel and stirred with medium agitation until a homogenous mixture was formed.
2. Silicone gum blend (KF-9030) was then added to the vessel, followed by amino modified Silicone fluid (DC 2-8566). The mixture was stirred with medium agitation further until a homogenous mixture formed.
3. Isononoyl Isononanoate was then added with stirring until a homoegenous mixture formed.
4. Fragrance was then added with stirring.

Compositions A made made according to the following protocol:
1. Cyclopentasiloxane and silicone fluid (300,000 cs) were added to a vessel and stirred with medium agitation until a homogenous mixture was formed.
2. Silicone gum blend (BY11-003) was added, followed by amino modified Silicone fluid (DC 2-8566) and the mixture was stirred with medium agitation further until a homogenous mixture formed.
3. Fragrance was then added with stirring.

The compositions are shown in the following Table 1:

TABLE 1
Compositions of Compositions 1, 2 and 3, in accordance with the
invention, and of Comparative Composition A.

| | | Amount | Amount | Amount | Amount |
|---|---|---|---|---|---|
| | | (wt %) | (wt %) | (wt %) | (wt %) |
| Supplier | Chemical name | A | 1 | 2 | 3 |
| | Cyclopentasiloxane | 80.7 | — | — | — |
| | (D5) | | | | |
| | Isododecane | — | 70.3 | 69.3 | 74.7 |
| | Isononyl | — | 1.3 | 1.4 | 1.0 |
| | Isononanoate | | | | |
| | Hydrogenated | — | 9.4 | 9.2 | 10.0 |
| | Polyisobutene | | | | |
| Dow Corning | Dimethicone | 10.0 | — | — | — |
| | (Gum 10%)/D5 | | | | |
| Shin-Etsu | Dimethicone | — | 6.4 | 14.0 | 4.8 |
| | (Gum 20%)/ | | | | |
| | Dimethicone 20 cs | | | | |
| Dow Corning | Dimethicone | 8.5 | 11.8 | 5.0 | 8.8 |
| | (300,000 cs) | | | | |
| Dow Corning | Amodimethicone | 0.5 | 0.5 | 0.5 | 0.5 |
| | (5,000 cs) | | | | |
| Perfume | Fragrance | <0.5 | <0.5 | <0.5 | <0.5 |

### Example 2

### Assessment of Sticky Feel During Use of Compositions 1, 2 and 3, in Accordance with the Invention, and Comparative Composition A

A half-head test was carried out under salon conditions, as follows:

The hair was twice pre-washed with a low-conditioning shampoo.

The hair was then parted in the middle of the head and combed.

One side of the head (wet hair) was treated with Composition A and the other with one of compositions 1, 2 or 3.

Each composition was assessed for stickiness during application to the hair.

The results are shown in Table 2 below.

TABLE 2
Stickiness during application to wet hair of
Compositions 1, 2 and 3, in accordance with
the invention, and Comparative Composition A.

| Attribute | 1 | 2 | 3 | A |
|---|---|---|---|---|
| Stickiness of hand | 8 | — | — | 28 |
| | — | 4 | — | 32 |
| | — | — | 4 | 32 |

A higher number indicates a higher level of stickiness.

The results shown are significantly different at 95% confidence level.

It will be seen that stickiness of hand is significantly lower for the compositions in accordance with the invention.

1. A hair treatment composition, comprising, by weight of the total composition:
a) from 60 to 80 wt % isododecane,
b) from 0.5 to 10 wt % isononyl isononanoate, and
c) from 5 to 30 wt % of hydrogenated polyisobutene.

1. A composition as claimed in claim 1, wherein the wt:wt ratio of isododecane:isononyl isononanoate is in the range of from 120:1 to 8:1.

3. A composition as claimed in claim 1, which further comprises a silicone, in an amount of from 1 to 15 wt %, wherein the silicone has a MWt in the range of from 500,000 cs to 20,000,000 cs.

4. A composition as claimed in claim 1, which further comprises a silicone, in an amount of from 5-15 wt %, wherein the silicone has a MWt in the range of 50,000cs-499,999cs.

5. A composition as claimed in claim 1, which further comprises an amino modified silicone, in an amount of from 0.01-5 wt %.

6. A hair treatment composition according to claim 1 which further comprises a further adjunct suitable for hair care.

7. A hair treatment composition according to claim 1 that is a serum or oil.

8. A hair treatment composition according to claim 1 that is a leave in composition.

9. A method of treating hair comprising the step of contacting hair with the composition defined in claim 1.

10. Use of a composition as defined in claim 1 to provide reduced stickiness of hand during use.
